(19) United States
(12) Patent Application Publication
(10) Pub. No.: US 2020/0399701 A1
(43) Pub. Date: Dec. 24, 2020

(54) SYSTEMS AND METHODS FOR USING DENSITY OF SINGLE NUCLEOTIDE VARIATIONS FOR THE VERIFICATION OF COPY NUMBER VARIATIONS IN HUMAN EMBRYOS

(71) Applicant: CooperSurgical, Inc., Trumbull, CT (US)

(72) Inventors: John BURKE, Reno, NV (US); Brian RHEES, Reno, NV (US); Joshua David BLAZEK, Houston, TX (US); Michael Jon LARGE, Houston, TX (US)

(21) Appl. No.: 16/906,441

(22) Filed: Jun. 19, 2020

Related U.S. Application Data
(60) Provisional application No. 62/865,126, filed on Jun. 21, 2019.

(51) Int. Cl. C12Q 1/6883; G16B 30/00

(52) U.S. Cl. CPC C12Q 1/6883; C12Q 2600/156; G16B 30/00

(57) ABSTRACT

A method for verifying a genomic variant region in an embryo, is disclosed. Embryo sequencing data is received by one or more processors. The received embryo sequencing data is aligned to a reference genome, by the one or more processors. A genomic variant region is identified in the aligned embryo sequencing data, by the one or more processors. A number of single nucleotide variants (SNVs) is counted in the identified genomic variant region, by the one or more processors. The counted number of SNVs in the identified genomic variant region is normalized against a baseline count of SNVs for a reference region corresponding to the identified genomic variant region to generate a normalized SNV density for the genomic variant region, by the one or more processors. The identified genomic variant region is verified, by the one or more processors, if the normalized SNV density in the identified genomic variant region satisfies a tolerance criterion.

### CROSS-REFERENCE TO RELATED APPLICATIONS

This application claims the benefit of priority to U.S. Provisional Patent Application 62/865,126 filed Jun. 21, 2019, which is incorporated herein by reference in its entirety.

### INCORPORATION BY REFERENCE

The disclosures of any patents, patent applications and publications cited herein are incorporated herein by reference in their entirety.

### FIELD

The embodiments disclosed herein are generally directed towards systems and methods for identifying copy number variation (CNV) in human embryos. More specifically, there is a need for systems and methods optimized for verifying CNVs calls made for human embryos prior to implantation into a mother.

### BACKGROUND

In vitro fertilization (IVF) is an assisted reproductive technology has become increasingly popular for women of advanced maternal age, couples with difficulties conceiving and as a means for facilitating gestational surrogacy. The process of fertilization involves extracting eggs, retrieving a sperm sample, and then manually combining an egg and sperm in a laboratory setting. The embryo(s) is then implanted in the host uterus to carry the embryo to term.

IVF procedures are expensive and can exact a significant emotional/physical toll on patients, so genetic screening of embryos prior to implantation is becoming an increasingly common for patients undergoing an IVF procedure. For example, currently IVF embryos are commonly screened for genetic abnormalities (e.g., CNV, SNV, etc.) and other conditions that can affect viability of transfer (i.e., embryo implantation viability). As with any diagnostic test, the accuracy of the resulting diagnosis is critical and that can be affected by a number of factors such as the data acquisition and analysis techniques used. In particular, bioinformatics analysis of genomic sequencing data with low coverage (˜0.1×) can result in the improper identification of segmental and mosaic aneuploidy and copy number variations (CNVs) due to sequencing artefact and noise in the sequencing data.

As such, there is a need for systems and methods that can independently verify the genetic abnormalities identified in an embryo.

### SUMMARY

This specification describes various exemplary embodiments systems and methods optimized for verifying CNVs calls made for human embryos prior to implantation into a mother.

In one aspect, a method for verifying a genomic variant region in an embryo, is disclosed. Embryo sequencing data is received by one or more processors. The received embryo sequencing data is aligned to a reference genome, by the one or more processors. A genomic variant region is identified in the aligned embryo sequencing data, by the one or more processors. A number of single nucleotide variants (SNVs) is counted in the identified genomic variant region, by the one or more processors. The counted number of SNVs in the identified genomic variant region is normalized against a baseline count of SNVs for a reference region corresponding to the identified genomic variant region to generate a normalized SNV density for the genomic variant region, by the one or more processors. The identified genomic variant region is verified, by the one or more processors, if the normalized SNV density in the identified genomic variant region satisfies a tolerance criterion.

In another aspect, a system for verifying a genomic variant region in an embryo, is disclosed. The system includes a data store, a computing device and a display. The data store is for storing embryo sequencing data. The computing device is communicatively connected to the data store and hosts an alignment engine, a genomic variant caller and a verification engine.

The alignment engine is configured to receive and align the embryo sequencing data against a reference genome. The genomic variant caller is configured to identify a genomic variant region in the aligned embryo sequencing data. The verification engine is configured to: count a number of single nucleotide variants (SNVs) in the identified genomic variant region and normalize the SNVs count in the identified genomic variant region against a baseline count of SNVs for a reference region corresponding to the identified genomic variant region to generate a normalized SNV density for the identified genomic variant region, and verify the identified genomic variant region if the normalized SNV density in the identified genomic variant region satisfies a tolerance criterion.

The display is communicatively connected to the computing device and configured to display a report containing genomic variant region results from the verification engine.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a graphical depiction of how total sequencing coverage normalized density correlations are better at detecting true biological changes in copy number (i.e., CNV) than correlations based on artifactual changes in sequencing coverage, in accordance with various embodiments.

FIG. 2 is a graphical depiction of the SNV density from a clinical embryo sample compared against a mean SNV density of 100 normal (non-CNV containing) embryo samples, in accordance with various embodiments.

FIG. 3 is a graphical representation of how SNV density can be used to confirm count-based CNV calls, in accordance with various embodiments.

FIG. 4 is an exemplary flowchart showing a method for verifying CNV calls made for an embryo, in accordance with various embodiments.

FIG. 5 is a schematic of a system for verifying CNV calls made for an embryo, in accordance with various embodiments.

FIG. 6 is a is a block diagram illustrating a computer system for use in performing the methods provided herein, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes various exemplary embodiment systems and methods optimized for verifying CNVs calls made for human embryos prior to implantation into a mother.

The disclosure, however, is not limited to these exemplary embodiments and applications or to the manner in which the exemplary embodiments and applications operate or are described herein.

Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

As used herein, the term “cell” is used interchangeably with the term “biological cell.” Non-limiting examples of biological cells include eukaryotic cells, plant cells, animal cells, such as mammalian cells, reptilian cells, avian cells, fish cells or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells and the like. A mammalian cell can be, for example, from a human, mouse, rat, horse, goat, sheep, cow, primate or the like.

A genome is the genetic material of a cell or organism, including animals, such as mammals, e.g., humans. In humans, the genome includes the total DNA, such as, for example, genes, noncoding DNA and mitochondrial DNA. The human genome typically contains 23 pairs of linear chromosomes: 22 pairs of autosomal chromosomes plus the sex-determining X and Y chromosomes. The 23 pairs of chromosomes include one copy from each parent. The DNA that makes up the chromosomes is referred to as chromosomal DNA and is present in the nucleus of human cells (nuclear DNA). Mitochondrial DNA is located in mitochondria as a circular chromosome, is inherited from only the female parent, and is often referred to as the mitochondrial genome as compared to the nuclear genome of DNA located in the nucleus.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

The term “read” with reference to nucleic acid sequencing refers to the sequence of nucleotides determined for a nucleic acid fragment that has been subjected to sequencing, such as, for example, NGS. Reads can be any a sequence of any number of nucleotides which defines the read length.

The phrase “sequencing coverage” or “sequence coverage,” used interchangeably herein, generally refers to the relation between sequence reads and a reference, such as, for example, the whole genome of cells or organisms, one locus in a genome or one nucleotide position in the genome. Coverage can be described in several forms (see, e.g., Sims et al. (2014) Nature Reviews Genetics 15:121-132). For example, coverage can refer to how much of the genome is being sequenced at the base pair level and can be calculated as NL/G in which N is the number of reads, L is the average read length, and G is the length, or number of bases, of the genome (the reference). For example, if a reference genome is 1000 Mbp and 100 million reads of an average length of 100 bp are sequenced, the redundancy of coverage would be 10×. Such coverage can be expressed as a “fold” such as 1×, 2×, 3×, etc. (or 1, 2, 3, etc. times coverage). Coverage can also refer to the redundancy of sequencing relative to a reference nucleic acid to describe how often a reference sequence is covered by reads, e.g., the number of times a single base at any given locus is read during sequencing. Thus, there may be some bases which are not covered and have a depth of 0 and some bases that are covered and have a depth of anywhere between, for example, 1 and 50. Redundancy of coverage provides an indication of the reliability of the sequence data and is also referred to as coverage depth. Redundancy of coverage can be described with respect to “raw” reads that have not been aligned to a reference or to aligned (e.g., mapped) reads. Coverage can also be considered in terms of the percentage of a reference (e.g., a genome) covered by reads. For example, if a reference genome is 10 Mbp and the sequence read data maps to 8 Mbp of the reference, the percentage of coverage would be 80%. Sequence coverage can also be described in terms of breadth of coverage which refers to the percentage of bases of a reference that are sequenced a given number of times at a certain depth.

As used herein, the phrase “low coverage” with respect to nucleic acid sequencing refers to sequencing coverage of less than about 10×, or about 0.001× to about 10×, or about 0.002× to about 0.2×, or about 0.01× to about 0.05×.

As used herein, the phrase “low depth” with respect to nucleic acid sequencing refers to sequencing depth of less than about 10×, or about 0.1× to about lox, or about 0.2× to about 5×, or about 0.5× to about 2×.

The term “resolution” with reference to genomic sequence nucleic acid sequence refers to the quality, or accuracy, and extent of the genomic nucleic acid sequence (e.g., sequence of the entire genome or a particular region or locus of the genome) obtained through nucleic acid sequencing of a cell(s), e.g., an embryo, or organism. The resolution of genomic nucleic acid sequence is primarily determined by the depth and breadth of coverage of the sequencing process and involves consideration of the number of unique bases that are read during sequencing and the number of times any one base is read during sequencing. The phrases “low resolution sequence” or “low resolution sequence data” or “sparse sequence data,” which are used interchangeably herein, with reference to genomic nucleic acid sequence of a cell(s), e.g., an embryo, or organism, refer to the nucleotide base sequence information of genomic nucleic acid that is obtained through low-coverage and low-breadth sequencing methods.

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation (CNV), inversion, etc.) which denotes a single or a grouping of genes (in DNA or RNA) that have undergone changes as referenced against a particular species or sub-populations within a particular species due to mutations, recombination/crossover or genetic drift. Genomic variants can be identified using a variety of techniques, including, but not limited to: array-based methods (e.g., DNA microarrays, etc.), real-time/digital/quantitative PCR instrument methods and whole or targeted nucleic acid sequencing systems (e.g., NGS systems, Capillary Electrophoresis systems, etc.). With nucleic acid sequencing, coverage data can be available at single base resolution.

The phrase “mosaic embryo” denotes embryos containing two or more cytogentically distinct cell lines. For example, a mosaic embryo can contain cell lines with different types of aneuploidy or a mixture of euploid and genetically abnormal cells containing DNA with genetic variants that may be deleterious to the viability of the embryo during pregnancy.

The phrase “SNV density” for a locus, where locus refers to a dynamic region of interest within a chromosome, refers to a value that is derived from the number of SNVs identified within the locus divided by the total number of sequence counts identified in that same locus for a sample.

### Nucleic Acid Sequence Data Generation

Some embodiments of the methods and systems provided herein for the analysis of genomic nucleic acids and classification of genomic features include analysis of nucleotide sequences of the genome of cells and/or organisms. Nucleic acid sequence data can be obtained using a variety of methods described herein and/or know in the art. In one example, sequences of genomic nucleic acid of cells, for example cells of an embryo, may be obtained from next-generation sequencing (NGS) of DNA samples extracted from the cells. NGS, also known as second-generation sequencing, is based on high-throughput, massively parallel sequencing technologies that involve sequencing of millions of nucleotides generated by nucleic acid amplification of samples of DNA (e.g., extracted from embryos) in parallel (see, e.g., Kulski (2016) “Next-Generation Sequencing—An Overview of the History, Tools and ‘Omic’ Applications,” in Next Generation Sequencing—Advances, Applications and Challenges, J. Kulski ed., London: Intech Open, pages 3-60).

Nucleic acid samples to be sequenced by NGS are obtained in a variety of ways, depending on the source of the sample. For example, human nucleic acids may readily be obtained via cheek brush swabs to collect cells from which nucleic acids are then extracted. In order to obtain optimum amounts of DNA for sequencing from embryos (for example, for pre-implantation genetic screening), cells (e.g., 5-7 cells) commonly are collected through trophectoderm biopsy during the blastocyst stage. DNA samples require processing, including, for example, fragmentation, amplification and adapter ligation prior to sequencing via NGS. Manipulations of the nucleic acids in such processing may introduce artifacts (e.g., GC bias associated with polymerase chain reaction (PCR) amplification), into the amplified sequences and limit the size of sequence reads. NGS methods and systems are thus associated with error rates that may differ between systems.

Additionally, software used in conjunction with identifying bases in a sequence read (e.g., base-calling) can affect the accuracy of sequence data from NGS sequencing. Such artifacts and limitations can make it difficult to sequence and map long repetitive regions of a genome and identify polymorphic alleles and aneuploidy in genomes. For example, because about 40% of the human genome is comprised of repeat DNA elements, shorter single reads of identical sequence that align to a repeat element in a reference genome often cannot be accurately mapped to a particular region of the genome. One way to address and possibly reduce some of the effects of errors and/or incompleteness in sequence determination is by increasing sequencing coverage or depth. However, increases in sequencing coverage are associated with increased sequencing times and costs. Paired-end sequencing can also be utilized, which increases accuracy in placement of sequence reads, e.g., in long repetitive regions, when mapping sequences to a genome or reference, and increases resolution of structural rearrangements such as gene deletions, insertions and inversions. For example, in some embodiments of methods provided herein, use of data obtained from paired-end NGS of nucleic acids from embryos increased read mapping by an average of 15%. Paired-end sequencing methods are known in the art and/or described herein and involve determining the sequence of a nucleic acid fragment in both directions (i.e., one read from one end of the fragment and a second read from the opposite end of the fragment). Paired-end sequencing also effectively increases sequencing coverage redundancy by doubling the number of reads and particularly increases coverage in difficult genomic regions.

### Nucleic Acid Sequence Analysis

In some embodiments of the methods and systems provided herein for the analysis of genomic nucleic acids and classification of genomic features, the sequences of nucleic acids obtained from cells, e.g., embryo cells, or organisms are used to reconstruct the genome (or portions of it) of the cells/organisms using methods of genomic mapping. Typically, genomic mapping involves matching sequences to a reference genome (e.g., a human genome) in a process referred to as alignment. Examples of human reference genomes that may be used in mapping processes include releases from the Genome Reference Consortium such as GRCh37 (hg19) released in 2009 and GRCh38 (hg38) released in 2013 (see, e.g., https://genome.ucsc.edu/cgi-bin/hgGateway?db=hg19 https://www.ncbi.nlm.nih.gov/assembly/GCF_000001405.39). Through alignment, sequence reads are assigned to genomic loci typically using computer programs to carry out the matching of sequences. Numerous alignment programs are publicly available and include Bowtie (see, e.g., http://bowtie-bio.sourceforge.net/manual.shtml) and BWA (see, e.g., http://bio-bwa.sourceforge.net/). Sequences that have been processed (for example to remove PCR duplicates and low-quality sequences) and matched to a locus are often referred to as aligned sequences or aligned reads.

In mapping of sequence reads to a genomic reference, it is possible to identify sequence nucleotide variants (SNV) or single nucleotide polymorphisms (SNP). It should also be noted that both the term SNV and SNP are used in accordance with various embodiments. Though both terms may be distinguishable to those of ordinary skill in the art, the terms can be used interchangeably in accordance with various embodiments herein. Thus, the use of either term should be inclusive of both terms as it applies to the process for analyzing received sequencing data. Single nucleotide variants/polymorphisms are the result of variation in the genome at a single nucleotide position. Several different NGS analysis programs for SNV detection are publicly available, known in the art and/or described herein. This method utilizes BCFTOOLS (open source) to digest aligned sequencing data and generate SNV/genotype calls used for the downstream processes. Detection and identification of genomic features, such as chromosomal abnormalities, e.g., aneuploidies, CNVs, through genome mapping of sequences from sample nucleic acids of cells or organisms presents particular challenges, particularly when sequence data is obtained from low-coverage and low-depth sequencing methods because the entire genome is not being interrogated, and what is being interrogated in the genome is particularly susceptible to bias and errors due to methodologies utilized to generate the sequencing data including, but not limited to: whole genome amplification, library preparation, and choice of next generation sequencing system and methodologies. Computer programs and systems are known in the art and/or described herein for increasing the ease and/or accuracy of interpretation of sequence data in identifying certain genomic features. For example, systems and methods for automated detection of chromosomal abnormalities including segmental duplications/deletions, mosaic features, aneuploidy and some forms of polyploidy are described in U.S. Patent Application Publication No. 2020/0111573 which is incorporated by reference herein. Such methods include de-noising/normalization (to de-noise raw sequence reads and normalize genomic sequence information to correct for locus effects) and machine learning and artificial intelligence to interpret (or decode) locus scores into karyograms. For example, after sequencing is completed, the raw sequence data is demultiplexed (attributed to a given sample), reads are aligned to a reference genome such as, e.g., HG19, and the total number of reads in each 1-million base pair bin is counted. This data is normalized based on GC content and depth and tested against a baseline generated from samples of known outcome. Statistical deviations from a copy number of 2 are then reported (if present, if not=euploid) as aneuploidy. Using this method, meiotic aneuploids and mitotic aneuploidy can be distinguished from each other based on the CNV metric. Based on the deviations from normal, a karyotype is generated with the total number of chromosomes present, any aneuploidies present, and the mosaic level (if applicable) of those aneuploidies.

Artifacts, variations in coverage and errors that can occur in NGS also present challenges in use of low-coverage sequencing data to accurately identify genomic variants. Therefore, there is a need for methods that can verify whether genomic variants identified from data obtained from low-coverage sequencing are in fact true genomic variants to ensure that they are correctly called.

Provided herein are improved, efficient, rapid, and cost-effective methods and systems for verifying genomic variant calls (in particular CNV calls) made using low-coverage sequencing data.

### Verification of CNV Calls Using SNV Density

The systems and methods, disclosed herein, involve using the determination that total sequencing coverage normalized density correlations are better at detecting true biological changes in copy number (i.e., CNV) than correlations based on artifactual changes in sequencing coverage. Historically, SNV density data has not been previously used to verify CNV calls at sequencing coverage levels less than 15×. In raw form, SNV density variability between different loci can often be greater than the variability due to copy number change. This shortcoming was addressed through the incorporation of a normalization step to smooth out SNV density variability between different loci, thus allowing SNV density to be used to verify CNV calls made with genomic sequencing data with low coverage. This is a significant improvement over conventional methods (which require data with sequencing coverage levels of 15× or more) as the higher the required sequencing coverage level, the more costly and time consuming (low throughput) the analysis.

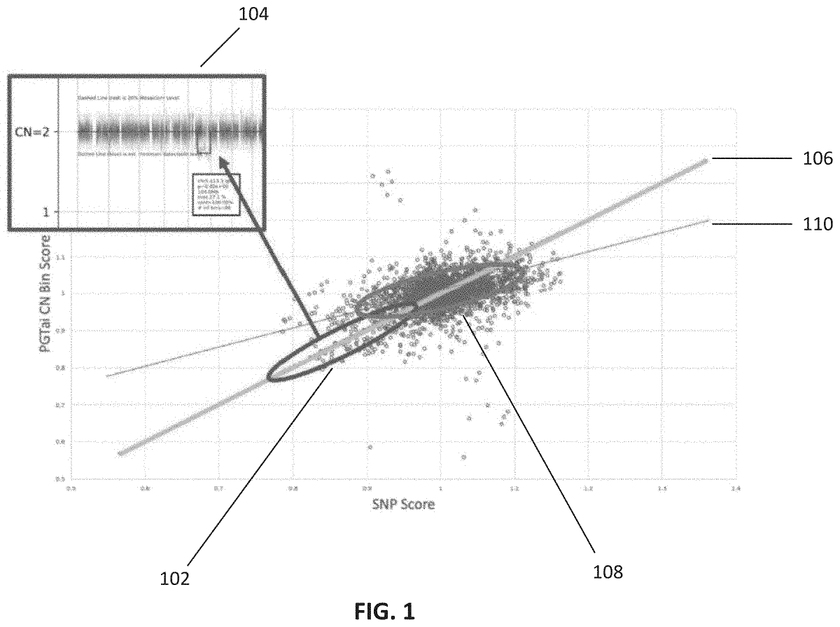

FIG. 1 is a graphical depiction of how total sequencing coverage normalized density correlations are better at detecting true biological changes in copy number (i.e., CNV) than correlations based on artifactual changes in sequencing coverage, in accordance with various embodiments.

As shown in FIG. 1, the read circle 102 represents the correlative relationship between total sequencing coverage normalized density when true biological changes are present in the embryo (and also observed in the CNV profile—see red arrow pointing to CNV profile 104). The correlation of the normalized CNV bin score (Y-axis) and the SNV density score for those individual bins (X-axis) as represented by the quasi-linear relationship represented by line 106 is higher than in when true biological changes are present as compared to when the signal is artifactual or noise as indicated by the CNV bins and their correlation with SNV density found in circle 108 and the subsequent trendline 110 with a reduced slope. The method thus leverages these correlation values between CNV bins score and SNV score when determining whether a change identified in the CNV method is verified by the method described in this disclosure.

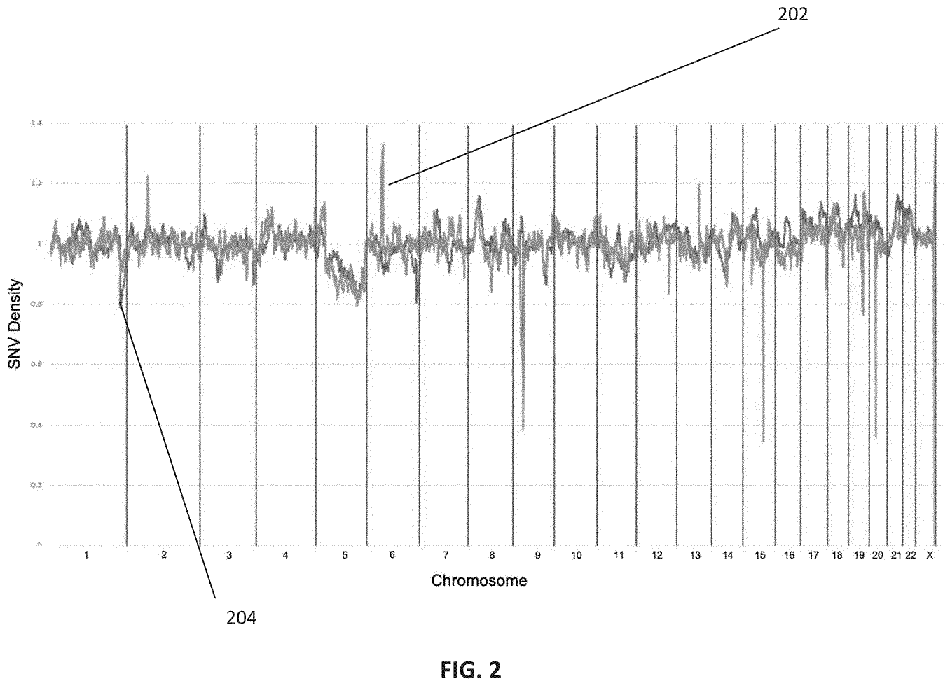

FIG. 2 is a graphical depiction of the SNV density from a clinical embryo sample 204 compared against a mean SNV density of 100 normal (non-CNV containing) embryo samples 202, in accordance with various embodiments.

The normalization operations disclosed herein exploits the fact that SNV density in samples with no CNV calls follow consistent patterns that can be used to normalize SNV density. Therefore, as shown in FIG. 2, the normalization of SNV density can involve dividing the SNV density 204 (derived from a clinical embryo sample) for a locus by the mean SNV density 202 in a baseline set of normal samples (i.e., 100 normal female embryos). This normalization function is shown in Equation 1.

Dₙₒᵣₘ(locus,baseline sample)=(Sample SNV Density at Locus)/(Average Baseline SNV Density at Locus) Equation 1:

The resulting normalized SNV density can then be used to confirm count-based CNV calls.

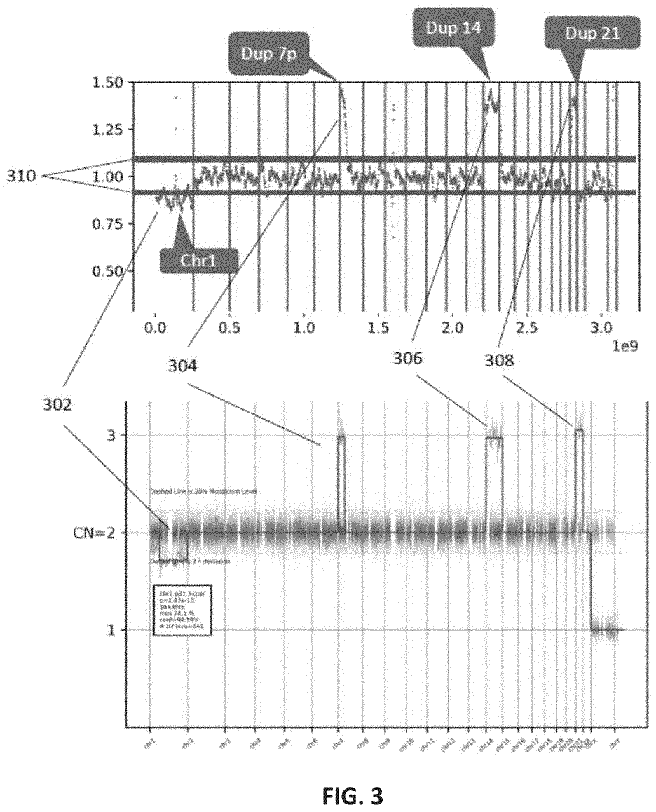

FIG. 3 is a graphical representation of how SNV density can be used to confirm count-based CNV calls, in accordance with various embodiments.

As shown in FIG. 3, potential CNV calls are made for chromosome 1 (deletion) 302, chromosome 7 (duplication) 304, chromosome 14 (duplication) 306, and chromosome 21 (duplication) 308 using count-based methods. These CNV calls were verified against a normalized SNV density graph, which includes a pre-set confidence interval used to verify whether the potential CNV calls are in fact real. In this instance, all four CNV calls were verified as real CNV calls because the graph shows that the SNV densities in the chromosome locations of the CNV calls fall outside the pre-set confidence interval.

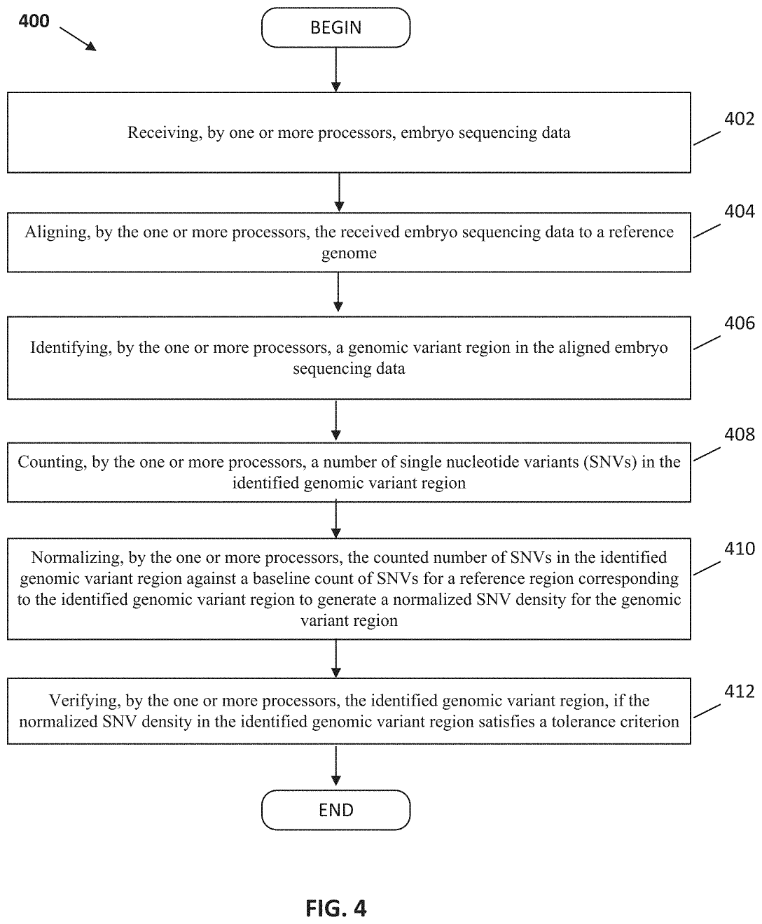

FIG. 4 is an exemplary flowchart showing a method for verifying CNV calls made for an embryo, in accordance with various embodiments.

In step 402, embryo sequencing data is received by one or more processors. In various embodiments, the embryo can be a human embryo. In various embodiments, the embryo is a non-human embryo.

In step 404, the received embryo sequencing data is aligned to a reference genome by the one or more processors. In various embodiments, the reference genome can be a whole genome obtained from a single individual. In various embodiments, the reference genome can be a composite whole genome from a plurality of individuals. Examples of reference genomes that can be used in the alignment process include, but are not limited to, genomes released from the Genome Reference Consortium such as GRCh37 (hg19) released in 2009 and GRCh38 (hg38) released in 2013 (see, e.g., https://genome.ucsc.edu/cgi-bin/hgGateway?db=hg19 https://www.ncbi.nlm.nih.gov/assembly/GCF_000001405.39).

In step 406, a genomic variant region in the aligned embryo sequencing data is identified by the one or more processors. In various embodiments, the genomic variant region is a CNV region identified using a count-based CNV calling method. In various embodiments, the genomic variant region is an aneuploidy region. In various embodiments, the genomic variant region is a polyploidy region. In various embodiments, the genomic variant region includes a sequence segment representing an entire chromosome. In various embodiments, the genomic variant region includes a sequence segment representing only part of a chromosome.

In step 408, the SNV the number of SNVs in the identified genomic variant region is counted by the one or more processors.

In step 410, the counted number of SNVs in the identified genomic variant region is normalized against a baseline count of SNVs for a reference region corresponding to the identified genomic variant region to generate a normalized SNV density for the genomic variant region by the one or more processors. In various embodiments, the baseline count of SNVs is obtained from sequencing data derived from one or more normal (non-CNV) sample(s). In various embodiments, the identified variant region and the reference region cover the same corresponding genome segment (or genome position). In various embodiments, the identified genomic variant region and the reference region include a sequence segment representing an entire chromosome. In various embodiments, the identified genomic variant region and the reference region includes a sequence segment representing only part of a chromosome.

In step 412, the identified genomic variant region is verified by the one or more processors if the normalized SNV density score in the identified genomic variant region satisfies a tolerance criterion. In various embodiments, if the SNV density for the identified genomic variant region is outside of the pre-set confidence interval of the average SNV density under the NULL hypothesis, there is no true copy number variation. In various embodiments, the pre-set confidence interval is about 90%. In various embodiments, the pre-set confidence interval is about 95%. In various embodiments, the pre-set confidence interval is about 96%, about 97%, about 98% and about 99%.

A duplication is verified if the SNV density is greater than the upper pre-set confidence limit and a deletion is verified if the SNV density is below the lower pre-set confidence limit. The pre-set confidence interval is defined according to a normality assumption (C±Z sigma/sqrt(N)), where C is the center or expected value of the average SNV density under the NULL hypothesis, N is the number of windows overlapping the identified genomic variant region, sigma is the global standard deviation of normalized SNV densities over all autosome chromosomes, and Z is the X th percentile of the standard normal distribution. The “+” symbol indicates that the values are added for the upper limit of the confidence interval and “−” symbol indicates subtraction for the lower limit of the confidence interval.

In various embodiments, the tolerance criterion is an expected SNV density for a reference region derived from a mosaic embryo.

In various embodiments, the identified genomic variant region is verified if its SNV density is above the lower (for duplications) or below the upper (for deletions) limits of the pre-set confidence interval of the alternate hypothesis of a mosaic embryo (containing a true copy number variation of mosaic level percentage m). In various embodiments, the pre-set confidence interval is about 90%. In various embodiments, the pre-set confidence interval is about 95%. In various embodiments, the pre-set confidence interval is about 96%, about 97%, about 98% and about 99%.

The pre-set confidence interval of the alternate hypothesis is defined according to normality assumption (C±Z sigma/sqrt(N)), where C is the center or expected value of the average SNV density under the alternate hypothesis, C=E(SNV density|m)=1.0±0.5*m/100, and, N is the number of windows overlapping the identified genomic variant region, sigma is the global standard deviation of normalized SNV densities over all autosome chromosomes, and Z is the X th percentile of the standard normal distribution. The “+” symbol indicates that the values are added for the upper limit of the confidence interval and the “−” symbol indicates subtraction for the lower limit of the confidence interval.

In various embodiments, the identified genomic variant region is verified if the identified genomic variant region includes a number of SNVs exceeds a preset variance number of SNVs over or under a baseline count of SNVs for the reference region.

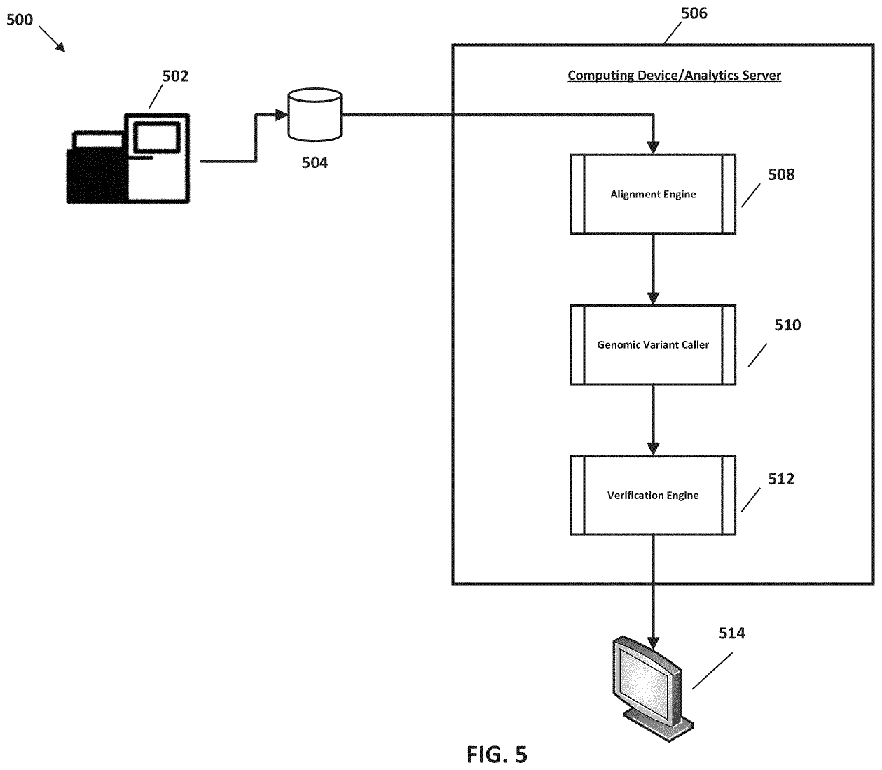

FIG. 5 is a schematic of a system for verifying CNV calls made for an embryo, in accordance with various embodiments.

System 500 includes a genomic sequencer 502, a data store 504, a computing device/analytics server 506 and a display 514.

The genomic sequence analyzer 502 can be communicatively connected to the data storage unit 504 by way of a serial bus (if both form an integrated instrument platform) or by way of a network connection (if both are distributed/separate devices). The genomic sequence analyzer 502 can be configured to process and analyze one or more genomic sequence datasets obtained from an embryo sample, which includes a plurality of fragment sequence reads. In various embodiments, the genomic sequence analyzer 902 can process and analyze one or more genomic sequence datasets that are generated by next-generation sequencing platforms and sequencers such as Llumina® sequencer, MiSeg™, NextSeg™ 500/550 (High Output), HiSeq 2500™ (Rapid Run), HiSeg™ 3000/4000, and NovaSeq.

In various embodiments, the processed and analyzed genomic sequence datasets can then be stored in the data storage unit 504 for subsequent processing. In various embodiments, one or more raw genomic sequence datasets can also be stored in the data storage unit 504 prior to processing and analyzing. Accordingly, in various embodiments, the data storage unit 504 is configured to store one or more genomic sequence datasets. In various embodiments, the processed and analyzed genomic sequence datasets can be fed to the computing device/analytics server 506 in real-time for further downstream analysis.

In various embodiments, the data storage unit 504 is communicatively connected to the computing device/analytics server 506. In various embodiments, the data storage unit 904 and the computing device/analytics server 506 can be part of an integrated apparatus. In various embodiments, the data storage unit 504 can be hosted by a different device than the computing device/analytics server 506. In various embodiments, the data storage unit 904 and the computing device/analytics server 506 can be part of a distributed network system. In various embodiments, the computing device/analytics server 506 can be communicatively connected to the data storage unit 504 via a network connection that can be either a “hardwired” physical network connection (e.g., Internet, LAN, WAN, VPN, etc.) or a wireless network connection (e.g., Wi-Fi, WLAN, etc.). In various embodiments, the computing device/analytics server 506 can be a workstation, mainframe computer, distributed computing node (part of a “cloud computing” or distributed networking system), personal computer, mobile device, etc.

In various embodiments, the computing device/analytics sever 506 can be configured to host an alignment engine 508, a genomic variant caller 510 and a verification engine 512.

The alignment engine 508 can be configured to receive and align the embryo sequencing data against a reference genome. In various embodiments, the reference genome can be a whole genome obtained from a single individual. In various embodiments, the reference genome can be a composite whole genome from a plurality of individuals. Examples of reference genomes that can be used in the alignment process include, but are not limited to, genomes released from the Genome Reference Consortium such as GRCh37 (hg19) released in 2009 and GRCh38 (hg38) released in 2013 (see, e.g., https://genome.ucsc.edu/cgi-bin/hgGateway?db=hg19 https://www.ncbi.nlm.nih.gov/assembly/GCF_000001405.39).

The genomic variant caller 510 can be configured to identify a genomic variant region in the aligned embryo sequencing data. In various embodiments, the genomic variant region is a CNV region identified using a count-based CNV calling method. In various embodiments, the genomic variant region is an aneuploidy region. In various embodiments, the genomic variant region is a polyploidy region. In various embodiments, the genomic variant region includes a sequence segment representing an entire chromosome. In various embodiments, the genomic variant region includes a sequence segment representing only part of a chromosome.

The verification engine 512 can be configured to count a number of single nucleotide variants (SNVs) in the identified genomic variant region and normalized the SNV count against a baseline count of SNVs for a reference region corresponding to the identified genomic variant region to generate a normalized SNV density for the identified genomic variant region and verify the identified genomic variant region if the SNV density in the identified genomic variant region satisfies a tolerance criterion.

In various embodiments, the baseline count of SNVs is obtained from sequencing data derived from one or more normal (non-CNV) sample(s). In various embodiments, the identified variant region and the reference region cover the same corresponding genome segment (or genome position). In various embodiments, the identified genomic variant region and the reference region include a sequence segment representing an entire chromosome. In various embodiments, the identified genomic variant region and the reference region includes a sequence segment representing only part of a chromosome.

In various embodiments, if the SNV density for the identified genomic variant region is outside of the pre-set confidence interval of the average SNV density under the NULL hypothesis, there is no true copy number variation. In various embodiments, the pre-set confidence interval is about 90%. In various embodiments, the pre-set confidence interval is about 95%. In various embodiments, the pre-set confidence interval is about 96%, about 97%, about 98% and about 99%.

A duplication is verified if the SNV density is greater than the upper pre-set confidence limit and a deletion is verified if the SNV density is below the lower pre-set confidence limit. The pre-set confidence interval is defined according to a normality assumption (C±Z sigma/sqrt(N)), where C is the center or expected value of the average SNV density under the NULL hypothesis, N is the number of windows overlapping the identified genomic variant region, sigma is the global standard deviation of normalized SNV densities over all autosome chromosomes, and Z is the X th percentile of the standard normal distribution. The “+” symbol indicates that the values are added for the upper limit of the confidence interval and “−” symbol indicates subtraction for the lower limit of the confidence interval.

In various embodiments, the tolerance criterion is an expected SNV density for a reference region derived from a mosaic embryo.

In various embodiments, the identified genomic variant region is verified if its SNV density is above the lower (for duplications) or below the upper (for deletions) limits of the pre-set confidence interval of the alternate hypothesis of a mosaic embryo (containing a true copy number variation of mosaic level percentage m). In various embodiments, the pre-set confidence interval is about 90%. In various embodiments, the pre-set confidence interval is about 95%. In various embodiments, the pre-set confidence interval is about 96%, about 97%, about 98% and about 99%.

The pre-set confidence interval of the alternate hypothesis is defined according to normality assumption (C±Z sigma/sqrt(N)), where C is the center or expected value of the average SNV density under the alternate hypothesis, C=E(SNV density|m)=1.0±0.5*m/100, and, N is the number of windows overlapping the identified genomic variant region, sigma is the global standard deviation of normalized SNV densities over all autosome chromosomes, and Z is the X th percentile of the standard normal distribution. The “+” symbol indicates that the values are added for the upper limit of the confidence interval and the “−” symbol indicates subtraction for the lower limit of the confidence interval.

In various embodiments, the identified genomic variant region is verified if the identified genomic variant region includes a number of SNVs exceeds a preset variance number of SNVs over or under a baseline count of SNVs for the reference region.

After the identified genomic variant region verification has been performed, the results can be displayed as a result or summary on a display or client terminal 514 that is communicatively connected to the computing device/analytics server 506. In various embodiments, the display or client terminal 514 can be a thin client computing device. In various embodiments, the display or client terminal 514 can be a personal computing device having a web browser (e.g., INTERNET EXPLORER™, FIREFOX™, SAFARI™, etc.) that can be used to control the operation of the genomic sequence analyzer 502, data store 504, alignment engine 508, genomic variant caller 510, and verification engine 512.

### Experimental Results

TABLE 1

| | True Positive | True Negative | False Positive | False Negative |
|---|---|---|---|---|
| Totals | 51 | 338 | 11 | 19 |

As shown above in Table 1, a total of 70 triploid samples and 349 diploid samples with known truth (SNP array) were interrogated by the methods, disclosed herein, for the presence or absence of female triploidy. The results are described above where “true positive” is defined as successful called disease state (polyploid), “true negative” is defined as successfully called “euploid” state, “false positive” is defined as incorrectly called disease state in a euploid embryo and “false negative” is defined as incorrectly called euploid in a disease state embryo.

The table clearly shows the high accuracy of the disclosed methods in verifying the existence of true CNVs in an embryo.

### Computer-Implemented System

In various embodiments, the methods for using density of SNVs for verification of CNVs in embryos can be implemented via computer software or hardware. That is, as depicted in FIG. 5, the methods disclosed herein can be implemented on a computing device/analytics server 506 that includes an alignment engine 508, a data store 504, a genomic variant caller 510, and a verification engine 512. In various embodiments, the computing device/analytics server 506 can be communicatively connected to a display device 514 via a direct connection or through an internet connection.

It should be appreciated that the various engines depicted in FIG. 5 can be combined or collapsed into a single engine, component or module, depending on the requirements of the particular application or system architecture. Moreover, in various embodiments, the Alignment Engine 508, data store 504, Genomic Variant Caller 510, and Verification Engine 512 can comprise additional engines or components as needed by the particular application or system architecture.

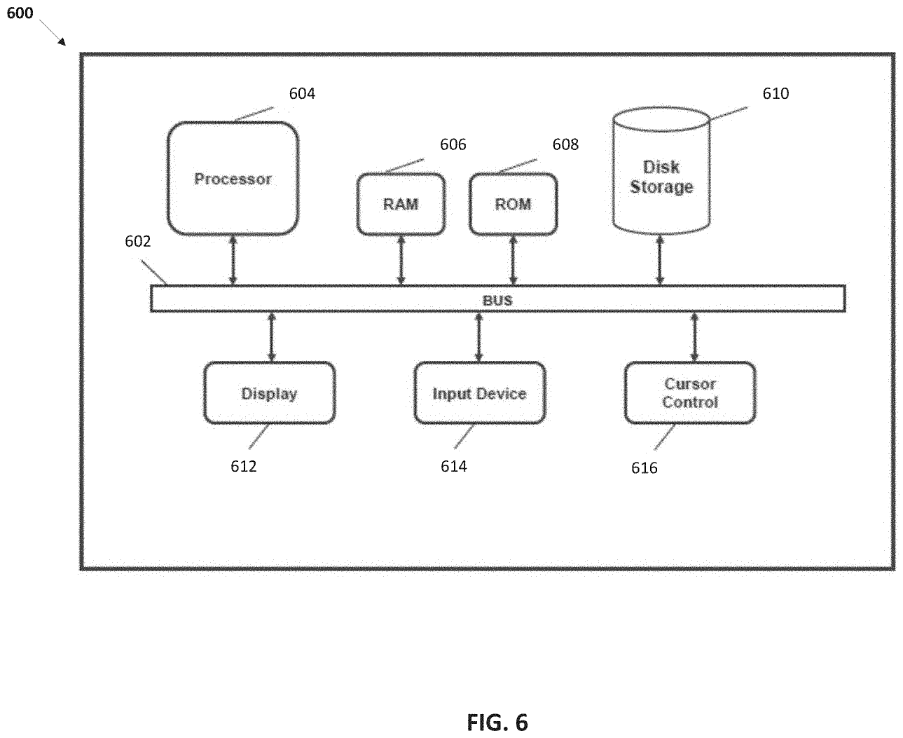

FIG. 6 is a block diagram that illustrates a computer system, in accordance with various embodiments. In various embodiments of the present teachings, computer system 600 can include a bus 602 or other communication mechanism for communicating information, and a processor 604 coupled with bus 602 for processing information. In various embodiments, computer system 600 can also include a memory, which can be a random access memory (RAM) 606 or other dynamic storage device, coupled to bus 602 for determining instructions to be executed by processor 604. Memory also can be used for storing temporary variables or other intermediate information during execution of instructions to be executed by processor 604. In various embodiments, computer system 600 can further include a read only memory (ROM) 608 or other static storage device coupled to bus 602 for storing static information and instructions for processor 604. A storage device 610, such as a magnetic disk or optical disk, can be provided and coupled to bus 602 for storing information and instructions.

In various embodiments, computer system 600 can be coupled via bus 602 to a display 612, such as a cathode ray tube (CRT) or liquid crystal display (LCD), for displaying information to a computer user. An input device 614, including alphanumeric and other keys, can be coupled to bus 602 for communicating information and command selections to processor 604. Another type of user input device is a cursor control 616, such as a mouse, a trackball or cursor direction keys for communicating direction information and command selections to processor 604 and for controlling cursor movement on display 612. This input device 614 typically has two degrees of freedom in two axes, a first axis (i.e., x) and a second axis (i.e., y), that allows the device to specify positions in a plane. However, it should be understood that input devices 614 allowing for 3 dimensional (x, y and z) cursor movement are also contemplated herein.

Consistent with certain implementations of the present teachings, results can be provided by computer system 600 in response to processor 604 executing one or more sequences of one or more instructions contained in memory 606. Such instructions can be read into memory 606 from another computer-readable medium or computer-readable storage medium, such as storage device 610. Execution of the sequences of instructions contained in memory 606 can cause processor 604 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 604 for execution. Such a medium can take many forms, including but not limited to, non-volatile media, volatile media, and transmission media. Examples of non-volatile media can include, but are not limited to, optical, solid state, magnetic disks, such as storage device 610. Examples of volatile media can include, but are not limited to, dynamic memory, such as memory 606. Examples of transmission media can include, but are not limited to, coaxial cables, copper wire, and fiber optics, including the wires that comprise bus 602.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 604 of computer system 600 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 600 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 600, whereby processor 604 would execute the analyses and determinations provided by these engines, subject to instructions provided by any one of, or a combination of, memory components 606/608/610 and user input provided via input device 614.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

What is claimed:

1. A method for verifying a genomic variant region in an embryo, comprising:
receiving, by one or more processors, embryo sequencing data;
aligning, by the one or more processors, the received embryo sequencing data to a reference genome;
identifying, by the one or more processors, a genomic variant region in the aligned embryo sequencing data;
counting, by the one or more processors, a number of single nucleotide variants (SNVs) in the identified genomic variant region;
normalizing, by the one or more processors, the counted number of SNVs in the identified genomic variant region against a baseline count of SNVs for a reference region corresponding to the identified genomic variant region to generate a normalized SNV density for the genomic variant region; and
verifying, by the one or more processors, the identified genomic variant region, if the normalized SNV density in the identified genomic variant region satisfies a tolerance criterion.

2. The method of claim 1, wherein the genomic variant region is a copy number variation region.

3. The method of claim 1, wherein the genomic variant region is an aneuploidy region.

4. The method of claim 1, wherein the genomic variant region is a polyploidy region.

5. The method of claim 1, wherein the reference region is an exact length of the identified genomic variant region.

6. The method of claim 1, wherein the reference region is derived from an euploid sample.

7. The method of claim 1, wherein the tolerance criterion is an expected SNV density for a reference region derived from an euploid embryo.

8. The method of claim 7, wherein the identified genomic variant region is verified if the normalized SNV density of the identified genomic variant region is greater or lesser than a pre-set confidence interval of the expected SNV density for the reference region.

9. The method of claim 8, wherein the lower pre-set confidence interval is 95%.

10. The method of claim 1, wherein the tolerance criterion is an expected SNV density for a reference region derived from a mosaic embryo.

11. The method of claim 10, wherein the identified genomic variant region is verified if the normalized SNV density of the identified genomic variant region is above a pre-set confidence interval of the expected SNV density for the reference region.

12. The method of claim 11, wherein the pre-set confidence interval 95%.

13. The method of claim 1, wherein the tolerance criterion is a preset variance number of SNVs over or under a baseline count of SNVs for the reference region.

14. A non-transitory computer-readable medium storing computer instruction for verifying a genomic variant region in an embryo, comprising:
receiving, by one or more processors, embryo sequencing data;
aligning, by the one or more processors, the received embryo sequencing data to a reference genome;
identifying, by the one or more processors, a genomic variant region in the aligned embryo sequencing data;
counting, by the one or more processors, a number of single nucleotide variants (SNVs) in the identified genomic variant region;
normalizing, by the one or more processors, the counted number of SNVs in the identified genomic variant region against a baseline count of SNVs for a reference region corresponding to the identified genomic variant region to generate a normalized SNV density for the genomic variant region; and
verifying, by the one or more processors, the identified genomic variant region, if the normalized SNV density in the identified genomic variant region satisfies a tolerance criterion.

15. A system for verifying a genomic variant region in an embryo, comprising:
a data store for storing embryo sequencing data;
a computing device communicatively connected to the data store, comprising,
an alignment engine configured to receive and align the embryo sequencing data against a reference genome,
a genomic variant caller configured to identify a genomic variant region in the aligned embryo sequencing data, and
a verification engine configured to:
count a number of single nucleotide variants (SNVs) in the identified genomic variant region and normalize the SNVs count in the identified genomic variant region against a baseline count of SNVs for a reference region corresponding to the identified genomic variant region to generate a normalized SNV density for the identified genomic variant region, and
verify the identified genomic variant region if the normalized SNV density in the identified genomic variant region satisfies a tolerance criterion; and
a display communicatively connected to the computing device and configured to display a report containing genomic variant region results from the verification engine.

16. The system of claim 15, wherein the genomic variant region is a copy number variation region.

17. The system of claim 15, wherein the genomic variant region is an aneuploidy region.

18. The system of claim 15, wherein the genomic variant region is a polyploidy region.

19. The system of claim 15, wherein the reference region is an exact length of the identified genomic variant region.

20. The system of claim 15, wherein the reference region is derived from an euploid sample.

21. The system of claim 15, wherein the tolerance criterion is an expected SNV density for a reference region derived from an euploid embryo.

22. The system of claim 21, wherein the identified genomic variant region is verified if the normalized SNV density of the identified genomic variant region is greater or lesser than a pre-set confidence interval of the expected SNV density for the reference region.

23. The system of claim 22, wherein the lower pre-set confidence interval is 95%.

24. The system of claim 15, wherein the tolerance criterion is an expected SNV density for a reference region derived from a mosaic embryo.

25. The system of claim 24, wherein the identified genomic variant region is verified if the normalized SNV density of the identified genomic variant region is above a pre-set confidence interval of the expected SNV density for the reference region.

26. The system of claim 25, wherein the pre-set confidence interval 95%.

27. The system of claim 15, wherein the tolerance criterion is a preset variance number of SNVs over or under a baseline count of SNVs for the reference region.
